(19) United States
(12) Patent Application Publication
(10) Pub. No.: US 2008/0097503 A1
(43) Pub. Date: Apr. 24, 2008

(54) Damping System for a Lancet Using Compressed Air

(76) Inventor: John P. Creaven, Granger, IN (US)

Correspondence Address: NIXON PEABODY LLP, 161 N. CLARK STREET, 48TH FLOOR, CHICAGO, IL, 60601, US

(21) Appl. No.: 11/660,835

(22) PCT Filed: Sep. 8, 2005
(86) PCT No.: PCT/US05/32167

§ 371 Date: Feb. 22, 2007

Related U.S. Application Data
(60) Provisional application No. 60/608,490, filed on Sep. 9, 2004.

(51) Int. Cl. A61B 5/151

(52) U.S. Cl. 606/182

(57) ABSTRACT

A lancing device comprises a cylinder, a plunger, a piston, a spring and an actuator. The cylinder includes a valve that is adapted to allow air to flow into the cylinder. The cylinder further forms an aperture. The plunger housing is adapted to seat a lancet therein. The piston is adapted to move within the cylinder. The piston is adapted to inhibit most of the air located within the cylinder from escaping between the cylinder and piston. The piston is attached to the plunger housing. The spring is located within the cylinder. The spring is located adjacent the piston opposite from the plunger housing. The actuator includes an inner shaft in which the inner shaft of the actuator extends into the cylinder through the aperture formed in the cylinder. The inner shaft is attached to the piston opposite from the plunger housing.

### CROSS-REFERENCE TO RELATED APPLICATION

This application claims priority to U.S. Provisional Application No. 60/608,490, filed on Sep. 9, 2004.

### FIELD OF THE INVENTION

The present invention relates generally to diagnostic instruments and, more particularly, to a system for damping a lancet using compressed air.

### BACKGROUND OF THE INVENTION

The quantitative determination of analytes in body fluids is of great importance in the diagnoses and maintenance of certain physiological abnormalities. For example, lactate, cholesterol and bilirubin should be monitored in certain individuals. In particular, determining glucose in body fluids is important to diabetic individuals who must frequently check the glucose level in their body fluids to regulate the glucose intake in their diets.

One method of obtaining a body fluid sample such as a whole blood sample is to use a lancing device. The whole blood sample may be used to monitor the glucose of an individual. Existing lancing devices use a lancet to pierce the tissue of the skin, allowing a blood sample to form on the skin's surface. The whole blood sample is then transferred to the testing device. The whole blood sample is often taken from the fingertips of a test subject for glucose monitoring because of the high concentration of capillaries that can provide an effective blood supply. Taking the blood from the fingertips, however, is disadvantageous because of the high concentration of nerve endings that cause pain and discomfort to many individuals.

In addition to the pain and discomfort inherent in piercing the fingertip, existing lancing devices may cause increased pain to many individuals by failing to properly dampen the lancet after initially piercing the skin. This may result in multiple punctures to the individual's skin, increasing the discomfort to the user. Additionally, existing lancing devices may fail to adequately guide the lancet into and out from an individual's skin. This results in non-linear puncturing and may give rise to a ripping of the skin, increasing the user's discomfort.

It would be desirable to have a lancing device and a method for using a lancing device that addresses these issues.

### SUMMARY OF THE INVENTION

A lancing device is disclosed according to one embodiment of the present invention. The lancing device includes an actuator, a cylinder, a plunger housing, a piston, and a spring. The actuator includes a head and an inner shaft, the inner shaft is attached at one end to the head of the actuator. The cylinder includes a one-way valve and an aperture. The one-way valve is adapted to allow air to flow into the cylinder. The plunger housing is adapted to seat a lancet therein. The piston is adapted to slide within the cylinder and to prohibit most of the air located within the cylinder from escaping between the cylinder and piston. The piston is adapted to attach to the plunger housing. The spring is located within the cylinder next to the piston opposite from the plunger housing. The inner shaft of the actuator extends into the cylinder through the aperture and the spring located within the cylinder. The inner shaft is adapted to attach to the piston opposite the head of the actuator.

A method for damping a lancet is disclosed according to one embodiment of the present invention. The method includes the act of compressing a spring from a resting position. The spring is attached to a piston. Both the spring and the piston are located within a cylinder. The spring is adapted to impart a force on the piston to move the piston in a first direction as the spring is compressed. The method includes the act of releasing the spring. The release allows the spring to expand. The expanding spring is adapted to move the piston in a second direction. The spring being further adapted to return to its resting position. The method further includes the act of forming a vacuum within the cylinder as the piston moves in the second direction. The method further includes the act of providing a valve adapted to allow air to enter the cylinder and relieve the vacuum formed within the cylinder as the piston moves in the second direction. The method further includes the act of preventing the air from escaping the cylinder as the spring returns to its resting position. The method further includes the act of compressing the air within the cylinder to slow the piston's movement in the first direction.

The above summary of the present invention is not intended to represent each embodiment, or every aspect, of the present invention. Additional features and benefits of the present invention are apparent from the detailed description, and figures set forth below.

### BRIEF DESCRIPTION OF THE DRAWINGS

FIG. 1a is a perspective view of a lancing device, according to one embodiment of the present invention.

FIG. 1b is an upper perspective view of the lancing device of FIG. 1a, according to one embodiment of the present invention.

FIG. 2 is a cross-sectional side view of the lancing device of FIG. 1a, without the lancet, in its resting position, according to one embodiment of the present invention.

FIG. 3 is a cross-sectional side view of the lancing device of FIG. 1a, without the lancet, in its ready position, according to one embodiment of the present invention.

FIG. 4 is a cross-sectional side view of the lancing device of FIG. 1a, without the lancet, in its extended position, according to one embodiment of the present invention.

### DESCRIPTION OF ILLUSTRATED EMBODIMENTS

The present invention is directed to a lancing device that is adapted to receive a lancet for use in drawing a body fluid from the skin. The body fluid generally contains at least one analyte that may then be examined to determine its concentration in the body fluid sample.

Lancing devices and lancets may be used to produce a blood or body fluid sample from a test subject. This sample may then be analyzed with a meter and test strip, or similar device, to determine the concentration of the analyte to be examined. Examples of the types of analytes which may be collected with a lancing device include glucose, lipid profiles (e.g., cholesterol, triglycerides, LDL and HDL), microalbumin, hemoglobin A1C, fructose, lactate, or bilirubin.

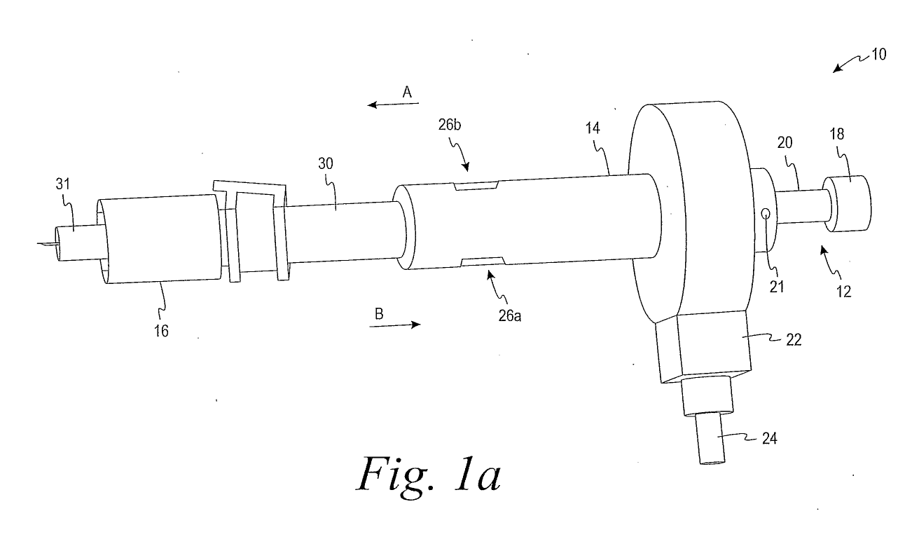

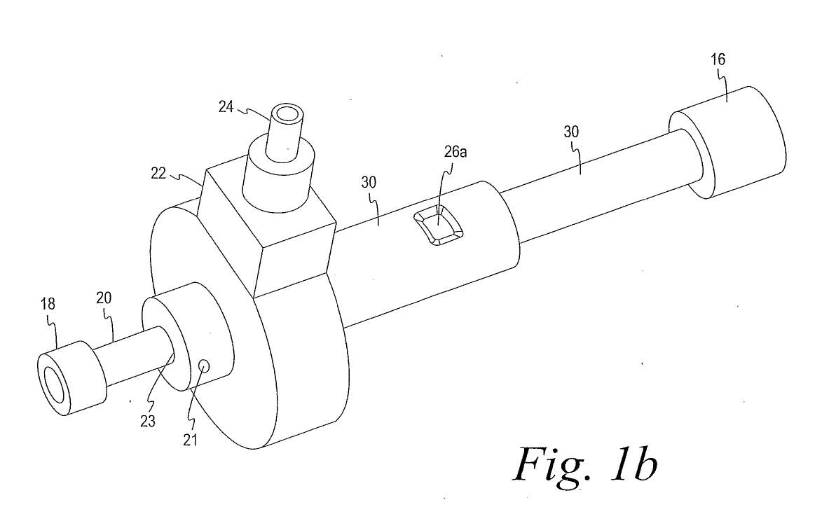

Turning now to the drawings and initially to FIGS. 1a-b, a lancing device 10 is shown according to one embodiment of the present invention. The lancing device 10 includes an actuator 12, a cylinder 14, and a plunger housing 16. The plunger housing 16 is adapted to seat a lancet 31 therein for puncturing the skin of a test subject. The actuator 12 includes a head 18 from which an inner shaft 20 extends. The inner shaft 20 extends into the cylinder 14 through an aperture 23 formed in the cylinder 14. The head 18 is shown as being generally circular, but it is contemplated that other shapes may be used. Additionally, according to certain embodiments the actuator 12 does not include a head 18 at all, but rather the inner shaft 20 is adapted to allow a user to actuate the lancing device 10.

The cylinder 14 includes an extension 22 extending from therefrom. A one-way-intake valve 24 is contained within a portion of the extension 22. The one-way valve 24 allows air to flow into the extension 22 and the cylinder 14. The cylinder 14 also contains at least one cylinder port. As illustrated, the cylinder 14 includes two cylinder ports 26a-b located on opposite sides of the cylinder 14.

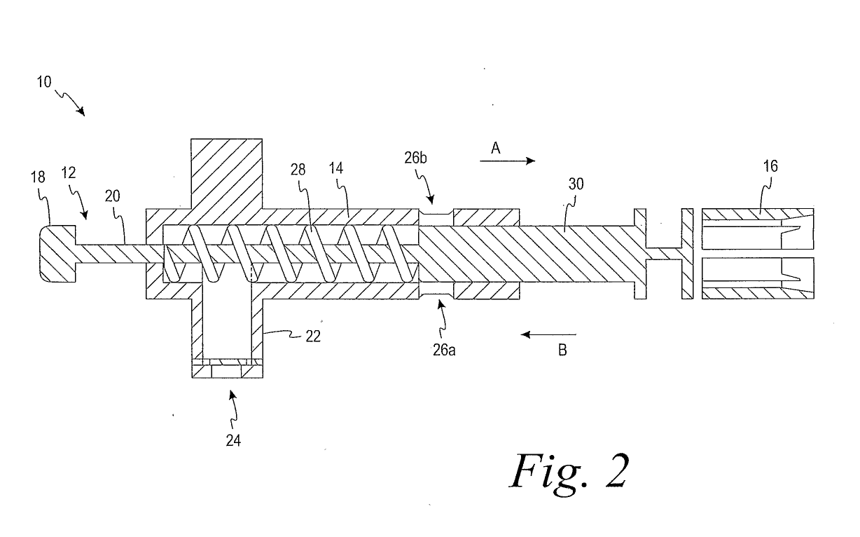

Referring now to FIG. 2, the lancing device 10 is shown in a resting position according to one embodiment of the present invention. As illustrated in FIG. 2, the inner shaft 20 extends into the cylinder 14 and through a spring 28. The inner shaft 20 is attached to a piston 30 opposite from the head 18. The piston 30 is attached to the plunger housing 16 opposite from the head 18. Thus, movement of the actuator 12 sets in motion both the piston 30 and the plunger housing 16 as well as causes the spring 28 to compress or extend.

The spring 28 has at least three positions a resting position, a ready position, and an extended position. The resting position is where the actuator 12 and piston 30 are in equilibrium with the spring 28 (when the actuator 12 is not locked in the firing mode). The ready position is where the spring 28 has been compressed by the actuator 12 prior to firing the device. This position may be achieved by completely compressing the spring 28 and/or compressing the spring 28 until the actuator 12 locks in a firing position. The extended position is where the spring 28 has fully extended after having been compressed and released. As should be understood, the resting position is between the ready position and the extended position.

The piston 30 is designed to move (e.g., slide, glide, skim, slip, skid, flow, etc.) within the cylinder 14 while maintaining sufficient contact with the cylinder 14 to inhibit most of the air located within the cylinder 14 and extension 22 from escaping between the cylinder 14 and piston 30. As illustrated in FIG. 2, when the lancing device 10 is in its resting position the piston 30 prevents or inhibits air from entering or exiting either of the cylinder ports 26a-b.

The piston 30 and cylinder 14 are shown as being generally circular, but it is contemplated that other shapes may be used. The cylinder 14 and piston 30 should be designed so as to create a snug fit between the piston 30 and cylinder 14 while allowing the piston 30 to move back-and-forth within the cylinder 14. For example, according to one embodiment, the cylinder 14 and piston 30 are generally rectangular in shape.

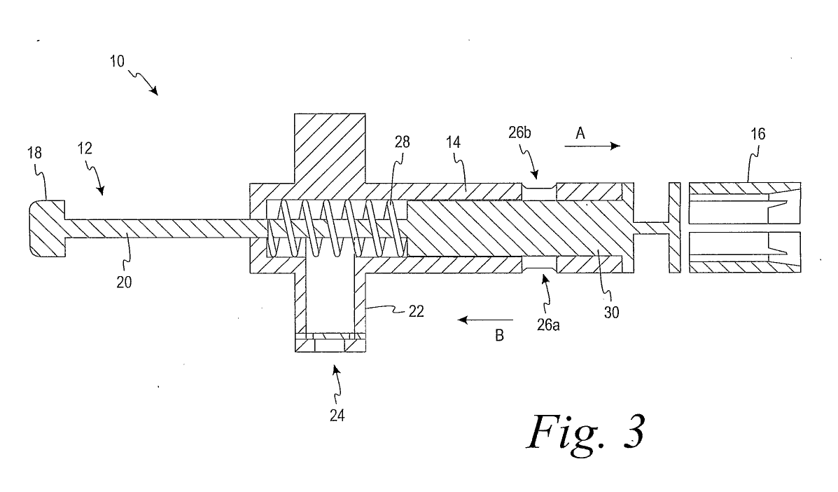

Turning now to FIG. 3, the lancing device 10 is shown in a ready position according to one embodiment of the present invention. As shown, the inner shaft 20 of the actuator 12 extends further from the cylinder 14 when the lancing device 10 is in the ready position. To cause the lancing device 10 to go from the resting position to ready position, the actuator 12 is moved in the direction of arrow B in FIG. 3 relative to the cylinder 14. As the actuator 12 is moved in the direction of arrow B, the piston 30 and the plunger housing 16 move in the B direction as well. The B direction movement of the piston 30 causes the spring 28 to compress. As the piston moves slowly in the B direction, the air trapped within the cylinder 14 and extension 22 seeps out between the cylinder 14 and the piston 30, as well as between the inner shaft 20 and the aperture 23.

Once the actuator 12 has been fully moved in the B direction, the lancing device 10 is in the ready position. In this position, the actuator 12 may be held in place by a firing mechanism (not shown). The firing mechanism may be used to allow the operator to actuate the lancing device 10 by depressing a firing button 21 (FIGS. 1a-b) separate from the actuator 12. Thus, a test subject may move the actuator into the ready position—and once the actuator 12 is held in place—reposition the lancing device 10 to the spot to be punctured. The test subject may then hold the lancing mechanism 10 still and depress the firing button 21 to cause the lancing device 10 to actuate, which when properly positioned, will cause the lancet 31 (FIG. 1a) to pierce the skin of the test subject.

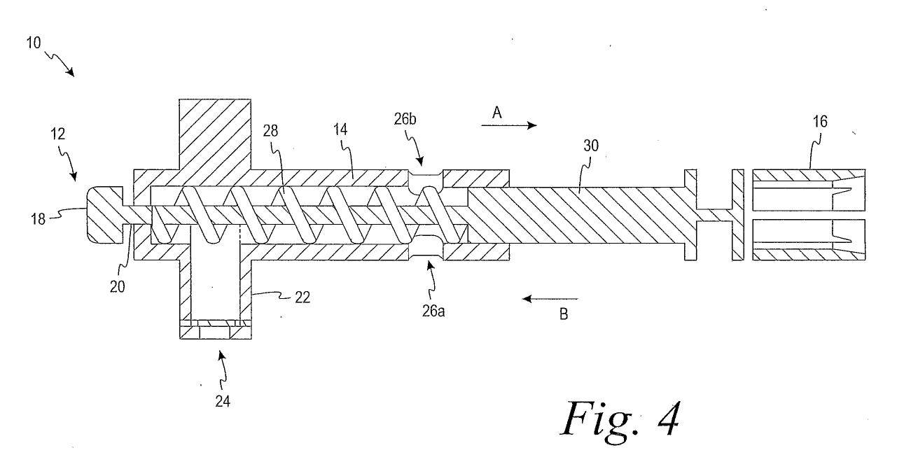

Turning now to FIG. 4, the lancing device 10 is shown with the plunger housing 16 and cylinder 14 fully extended after the lancing device 10 has been actuated. When the lancing device is actuated, the spring 28 begins to expand from its compressed state (shown in FIG. 3) until it is completely expanded as shown in FIG. 4. The expansion of the spring 28 forces the piston 30 to extend out of the cylinder 14 in the direction of arrow A in FIG. 4, which causes the plunger housing 16 to move away from the cylinder 14. As the plunger housing 16 moves away from the cylinder 14, the lancet 31 (FIG. 1a) seated in the plunger housing 16 pierces the skin of a test subject.

Referring again to FIGS. 3-4, the damping mechanism for the lancing device 10 will be described in more detail. As the actuator 12 is pulled in the direction of arrow B, air slowly escapes from the cylinder 14 and the spring 28 is compressed. Once the device 10 is fired, the spring 28 expands forcing the piston 30 to extend out or the cylinder 14. As the piston 30 moves in the direction of arrow A, a vacuum is formed within the cylinder 14 and extension 22. This vacuum causes air to enter through the one-way valve 24 into the cylinder 14 and extension 22 to fill the void. Air continues to enter through the valve 24 as the piston 30 extends from the cylinder 14. Once the piston 30 clears the cylinder ports 26a-b, however, the ports 26a-b allow air to enter into the cylinder 14 and the extension 22. As this air is allowed to enter, the vacuum within the cylinder 14 is relieved and the valve 24 begins to close.

The momentum of the piston 30—and the spring force previously created by extending the actuator 12 in the B direction—causes the spring 28 to pass the resting position of the spring 28 and near the extended position. Eventually, the return force on the spring 28 slows the extension of the piston 30 until the piston 30 comes to rest. The return force of the spring 28 then causes the piston 30 to move back in the B direction and further insert into the cylinder 14. As the piston 30 begins to return, air from the cylinder 14 is initially allowed to escape from the cylinder ports 26a-b. Once the piston 30 reaches the cylinder ports 26a-b, however, air is no longer able to escape from these cylinder ports 26a-b and the pressure builds within the cylinder 14 and the extension 22. As the pressure increases, the one-way valve 24 closes, preventing the air from escaping the cylinder 14 or extension 22. Thus, the air is trapped within the cylinder 14 and extension 22.

As the piston 30 continues to move back into the cylinder 14, the air further compresses within the cylinder 14 causing the piston 30 to slow. This continues until the piston 30 is stopped by the compression of the air, at which point, the spring 28 has recompressed slightly. Once the piston 30 stops, the spring force again takes over and re-extends the piston 30 slightly in the A direction. As the piston 30 re-extends, air is allowed to enter the cylinder 14 and the extension 22 through the one-way valve 24. Once the piston 30 begins to move in the B direction again, the valve 24 closes and the air within the cylinder 14 and extension 22 compresses causing the piston 30 to further slow. This process continues until the spring 28 reaches its resting (or equilibrium) point, wherein the lancing device 10 reaches its resting position as shown in FIG. 2.

By utilizing a compressed air damping system, the spring 28 is prevented or inhibited from greatly over-compressing as the piston 30 is returned back into the cylinder 14. The slightly recompressed spring 28 does not cause the piston 30 to re-extend sufficiently to repuncture the test subjects skin. Thus, by utilizing the compressed air damping system multiple punctures to the test subject—and the pain associated therewith—is prevented or inhibited.

Because of the rapid damping associated with using compressed air as described, the design of the piston 30 and lancing device 10 is afforded greater flexibility. The damping system of the present invention allows an elongated piston 30 to be utilized because of the shortened distance required to slow the piston 30. The use of an elongated piston allows a greater portion of the piston to remain within the cylinder as the test subject's skin is punctured. This reduces the side-to-side motion of the piston, the attached plunger housing, and the lancet inserted in the plunger housing. Thus, a more linear puncture may be achieved, which assists in preventing or inhibiting the tearing of a test subject's skin.

According to one embodiment of the present invention, the lancing device 10 does not include cylinder ports 26a-b. In this embodiment, the return of the spring 28 from its extended position to its resting position is retarded even further because the air begins to compress immediately upon the return of the spring 28.

### Alternative Embodiment A

A lancing device comprising:

a cylinder including a valve, the valve being adapted to allow air to flow into the cylinder, the cylinder further forming an aperture;

a plunger housing adapted to seat a lancet therein;

a piston adapted to move within the cylinder, the piston being adapted to inhibit most of the air located within the cylinder from escaping between the cylinder and piston, the piston being attached to the plunger housing;

a spring located within the cylinder, the spring being located adjacent the piston opposite from the plunger housing; and

an actuator including an inner shaft, wherein the inner shaft of the actuator extends into the cylinder through the aperture formed in the cylinder, the inner shaft being attached to the piston opposite from the plunger housing.

### Alternative Embodiment B

The lancing device according to embodiment A wherein the cylinder includes an extension that extends therefrom, the one-way-intake valve being located within a portion of the extension.

### Alternative Embodiment C

The lancing device according to embodiment A, the lancing device further comprising a lancet adapted to pierce the skin of a test subject.

### Alternative Embodiment D

The lancing device according to embodiment A, wherein the valve is a one-way valve.

### Alternative Embodiment E

The lancing device according to embodiment A, wherein the cylinder further includes at least one cylinder port, the at least one cylinder port being adapted to allow air to flow into and out-from the cylinder.

### Alternative Embodiment F

The lancing device according to embodiment E, wherein the cylinder further includes a second cylinder port, the second cylinder port being adapted to allow air to flow into and out-from the cylinder.

### Alternative Embodiment G

The lancing device according to embodiment F, wherein the at least one cylinder port and the second cylinder port are located approximately opposite each other with respect to the cylinder.

### Alternative Embodiment H

The lancing device according to embodiment A wherein the actuator is adapted to compress the spring when a force is applied in a direction opposite the spring.

### Alternative Process I

A method for using a lancing device, the method comprising the acts of: providing a lancet device including,

(i) a cylinder including a valve, the valve being adapted to allow air to flow into the cylinder, the cylinder further forming an aperture,

(ii) a plunger housing adapted to seat a lancet therein,

(iii) a piston adapted to move within the cylinder, the piston being attached to the plunger housing,

(iv) a spring located within the cylinder, the spring being located adjacent the piston opposite from the plunger housing, the spring being adapted to move the piston in a first direction and a second direction, the spring having at least a resting position, a ready position, and an extended position, the ready position being between the ready position and the extended position, and

(v) an actuator including an inner shaft, wherein the inner shaft of the actuator extends into the cylinder through the aperture formed in the cylinder, the inner shaft being attached to the piston opposite from the plunger housing;

compressing the spring from the resting position into the ready position;

releasing the spring, the release allowing the spring to expand, the expanding spring imparting a firing force on the piston in the first direction until reaching the extended position of the spring, wherein the spring imparts a return force on the piston in the second direction until reaching the resting position of the spring;

forming a vacuum within the cylinder as the piston moves in the first direction;

allowing air to enter the cylinder through the valve and relieve the vacuum formed within the cylinder as the piston moves in the first direction;

inhibiting the air from escaping the cylinder as the spring returns to its resting position and the piston moves in the second direction;

compressing the air within the cylinder to slow the piston's movement in the second direction.

### Alternative Process J

The method according to process I, wherein the plunger housing includes a lancet.

### Alternative Process K

The method according to process J further comprising the act of piercing a skin of a test subject with the lancet prior to compressing the air within the cylinder.

### Alternative Process L

The method according to process I wherein the cylinder is provided with at least one cylinder port, the cylinder port being adapted to allow air to enter the cylinder prior to compressing the air within the cylinder.

### Alternative Process M

The method according to process I further comprising inhibiting the side-to-side movement of the piston with the cylinder.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention as defined by the appended claims.

1. A lancing device comprising:
a cylinder including a valve, the valve being adapted to allow air to flow into the cylinder, the cylinder further forming an aperture;
a plunger housing adapted to seat a lancet therein;
a piston adapted to move within the cylinder, the piston being adapted to inhibit most of the air located within the cylinder from escaping between the cylinder and piston, the piston being attached to the plunger housing;
a spring located within the cylinder, the spring being located adjacent the piston opposite from the plunger housing; and
an actuator including an inner shaft, wherein the inner shaft of the actuator extends into the cylinder through the aperture formed in the cylinder, the inner shaft being attached to the piston opposite from the plunger housing.

2. The lancing device according to claim 1, wherein the cylinder includes an extension that extends therefrom, the one-way-intake valve being located within a portion of the extension.

3. The lancing device according to claim 1, the lancing device further comprising a lancet adapted to pierce the skin of a test subject.

4. The lancing device according to embodiment A, wherein the valve is a one-way valve.

5. The lancing device according to claim 1, wherein the cylinder further includes at least one cylinder port, the at least one cylinder port being adapted to allow air to flow into and out-from the cylinder.

6. The lancing device according to claim 5, wherein the cylinder further includes a second cylinder port, the second cylinder port being adapted to allow air to flow into and out-from the cylinder.

7. The lancing device according to claim 6, wherein the at least one cylinder port and the second cylinder port are located approximately opposite each other with respect to the cylinder.

8. The lancing device according to claim 1 wherein the actuator is adapted to compress the spring when a force is applied in a direction opposite the spring.

9. A method for using a lancing device, the method comprising the acts of:
providing a lancet device including,
(i) a cylinder including a valve, the valve being adapted to allow air to flow into the cylinder, the cylinder further forming an aperture,
(ii) a plunger housing adapted to seat a lancet therein,
(iii) a piston adapted to move within the cylinder, the piston being attached to the plunger housing,
(iv) a spring located within the cylinder, the spring being located adjacent the piston opposite from the plunger housing, the spring being adapted to move the piston in a first direction and a second direction, the spring having at least a resting position, a ready position, and an extended position, the ready position being between the ready position and the extended position, and
(v) an actuator including an inner shaft, wherein the inner shaft of the actuator extends into the cylinder through the aperture formed in the cylinder, the inner shaft being attached to the piston opposite from the plunger housing;
compressing the spring from the resting position into the ready position;
releasing the spring, the release allowing the spring to expand, the expanding spring imparting a firing force on the piston in the first direction until reaching the extended position of the spring, wherein the spring imparts a return force on the piston in the second direction until reaching the resting position of the spring;
forming a vacuum within the cylinder as the piston moves in the first direction;
allowing air to enter the cylinder through the valve and relieve the vacuum formed within the cylinder as the piston moves in the first direction;
inhibiting the air from escaping the cylinder as the spring returns to its resting position and the piston moves in the second direction;
compressing the air within the cylinder to slow the piston's movement in the second direction.

10. The method according to claim 9, wherein the plunger housing includes a lancet.

11. The method according to claim 10, further comprising the act of piercing a skin of a test subject with the lancet prior to compressing the air within the cylinder.

12. The method according to claim 9, wherein the cylinder is provided with at least one cylinder port, the cylinder port being adapted to allow air to enter the cylinder prior to compressing the air within the cylinder.

13. The method according to claim 9, further comprising inhibiting the side-to-side movement of the piston with the cylinder.
